(19) United States
(12) Patent Application Publication
(10) Pub. No.: US 2012/0048271 A1
(43) Pub. Date: Mar. 1, 2012

(54) SOLID DOSAGE AT PATIENT INTERFACE

(76) Inventors: Kevin Peter O'Donnell, Auckland (NZ); Dean Antony Barker, Auckland (NZ)

(21) Appl. No.: 13/217,102

(22) Filed: Aug. 24, 2011

Related U.S. Application Data
(60) Provisional application No. 61/377,235, filed on Aug. 26, 2010.

(51) Int. Cl. A61M 16/16

(52) U.S. Cl. 128/203.16

(57) ABSTRACT

The present invention relates to an interface for delivering salt or other medicament to a patient. The interface has a receptacle for medicament in solid form and an inlet adapted to couple directly or indirectly to a source of a heated and humidified flow of gas. The inlet is arranged to direct an incoming heated and humidified flow of gas to the receptacle. An outlet is arranged to receive and deliver a heated and humidified flow of gas from the receptacle to the patient's airway.

### CROSS-REFERENCE TO RELATED APPLICATIONS

This application claims the benefit of priority under 35 U.S.C. §119(e) of U.S. Provisional Patent Application No. 61/377,235, filed Aug. 26, 2010, the disclosure of which is hereby incorporated by reference in its entirety.

### BACKGROUND OF THE INVENTION

1. Field of the Invention

This invention relates to an apparatus and method of providing a medicament to a patient as part of respiratory therapy.

2. Description of the Related Art

A number of methods are known in the art for assisting a patient's breathing. Continuous Positive Airway Pressure (CPAP) involves the administration of air under pressure to a patient, usually by a patient interface such as a nasal mask. It is used in the treatment of snoring and Obstructive Sleep Apnoea (OSA), a condition characterised by repetitive collapse of the upper airway during inspiration. Positive pressure splints the upper airway open, preventing its collapse. Treatment of OSA with nasal CPAP has proven to be both effective and safe.

CPAP is also commonly used for patients with a variety of respiratory illnesses, including Chronic Obstructive Pulmonary Disease (COPD). Upper airway symptoms adversely affect treatment with CPAP. Mucosal drying is uncomfortable and may awaken patients during the night. Therefore, the use of CPAP with humidifiers has been widely used.

Breathing air found in subterranean salt chambers has long been used as the basis for the treatment of respiratory diseases. Inhalation of airborne salt particles, a practise known as speleotherapy (“Horvath T, “Speleotherapy: a special kind of climatotherapy, its role in respiratory rehabilitation”, Disability & Rehabilitation, 1986; 8(2):90-92”) is well known through parts of central and eastern Europe. The replication of this treatment using direct inhalation of salt particles by a patient is also well known, a treatment method often referred to as halotherapy (Cervinskaya A V, Zilber N A, “Halotherapy for Treatment of Respiratory Diseases”, Journal of Aerosol Medicine, 1995 Jan. 1; 8(3):221-232 and A. V. Chervinskaia, “The scientific validation and outlook for the practical use of halo-aerosol therapy”. Voprosy kurortologii, fizioterapii, i lechebno fizichesko kultury (1):21).

More recent applications of salt based therapies for respiratory treatment in patients have been investigated, of which those associated with the condition of cystic fibrosis seem to be finding the most success, whilst others, including treatment of asthma (Baldini et al, “Speleotherapy in asthmatic children: improvement of allergic inflammation parameters”, Annals of Allergy, Asthma and Immunology, 1998; 8079 and Beamon S et al “Speleotherapy for asthma”, Cochrane database of systematic reviews, 2001; (2)) and COPD seem to be falling out of favour with developing medical opinion.

Some proponents of salt inhalation therapies would claim proof of efficacy from clinical trials of halotherapy for the treatment of asthma, chronic bronchitis, and a range of upper and lower respiratory tract diseases. Mainstream medical and scientific opinion suggests that the credibility of efficacy across all of these areas is exaggerated (Bennett W D, “Salt: Good for What Ails the Airways?”, Journal of Aerosol Medicine, Volume 8, Number 3, 1995), but there is optimism of the efficacy of the use of salt based treatments for those suffering from cystic fibrosis (O'Sullivan B P, Freedman S D, “Cystic fibrosis”, The Lancet, 2009 May 30; 373(9678):1891-1904). In clinical trials, these treatments have usually been administered as a saline aerosol to be inhaled by the patient (Eng P et al, “Short-term efficacy of ultrasonically nebulized hypertonic saline in cystic fibrosis”, Pediatr Pulmonol, 1996; 21(2):77-83 and Wark P, McDonald V M. Nebulised hypertonic saline for cystic fibrosis. Cochrane Database of Systematic Reviews 2009, Issue 2. Art. No.: CD001506. DOI:10.1002/14651858.CD001506.pub3.) Further affiliations such as for the treatment of bronchiolitis in infants (Zhang L, Mendoza-Sassi R A, Wainwright C, Klassen T P. Nebulized hypertonic saline solution for acute bronchiolitis in infants. Cochrane Database of Systematic Reviews 2008, Issue 4. Art. No.: CD006458. DOI:10.1002/14651858.CD006458.pub2.)

Administration of dry powder forms of medicaments are disclosed in U.S. Pat. No. 5,881,720 of The Procter & Gamble Company and U.S. Pat. No. 7,318,435 of Dimitrious Pentafragas.

A system and method of delivering medications where an infuser is used is disclosed in US2008/0072899 of Vapotherm. This patent application envisages the delivery of saline solutions including delivery by cannula.

US2009/0104272 of Parion Sciences, Inc discloses a method of treating COPD by administering an effective amount of an osmolyte (including active salts, for example) by a nebuliser connected to a nasal cannula.

### SUMMARY OF THE INVENTION

It is an object of the present invention to provide a medicament to a patient as part of respiratory therapy.

In one aspect the present invention may be said to consist in an interface for delivering salt to a patient, the interface comprising: a receptacle for salt in solid form, an inlet adapted to couple directly or indirectly to a source of a heated and humidified flow of gas, the inlet being arranged to direct an incoming heated and humidified flow of gas to the receptacle, and an outlet arranged to receive and deliver a heated and humidified flow of gas from the receptacle to the patient's airway.

Preferably, in use, the receptacle contains salt in solid form and the inlet directs incoming heated and humidified flow of gas to contact a surface of the salt in solid form to uptake salt into the flow of gas.

Preferably the gas flow uptakes salt by elution.

Preferably upon uptake of salt, the heated and humidified flow of gas from the receptacle comprises eluted salt resulting in a salt laden gas.

Preferably the gas is heated to between 30-40 degrees C.

Preferably the flow rate of the gas is between 5-50 litres/minute.

Preferably the humidity of the gas is between 80%-100%.

Preferably the interface is one of a cannula and a tracheotomy connection.

Preferably the receptacle is in or proximate the outlet.

In another aspect the present invention may be said to consist in a method of providing salt therapy comprising: passing a humidified and heated flow of gas to contact a surface of a salt in solid form disposed at or near a patient to uptake salt into the flow of gas, and delivering the salt laden flow of gas to a patient.

Preferably the method treats COPD and/or Cystic Fibrosis

Preferably the gas is heated to between 30-40 degrees C.

Preferably the flow rate of the gas is between 5-50 litres/minute.

Preferably the humidity of the gas is between 80%-100%.

Preferably the gas flow uptakes salt by elution.

In another aspect the present invention may be said to consist in a breathing assistance apparatus for delivering salt therapy to a patient with a flow of gas comprising: a gases supply providing a flow of gas, a humidifier and heater for heating and humidifying a flow of gas from the gases supply, a patient interface according to claim 1 or 25 coupled to the gases supply via an inspiratory conduit to deliver a heated and humidified flow of gas from the gases supply to a patient.

Preferably, in use, the receptacle contains salt in solid form and the inlet directs incoming heated and humidified flow of gas to contact a surface of the salt in solid form to uptake salt into the flow of gas.

Preferably the gas flow uptakes salt by elution.

Preferably upon uptake of salt, the heated and humidified flow of gas from the receptacle comprises eluted salt resulting in a salt laden gas.

Preferably the gas is heated to between 30-40 degrees C.

Preferably the flow rate of the gas is between 5-50 litres/minute.

Preferably the humidity of the gas is between 80%-100%.

Preferably the interface is one of a cannula or a tracheotomy connection.

Preferably the receptacle is in or proximate the outlet.

Preferably the interface comprises an inlet adapted to couple directly or indirectly to a source of a heated and humidified flow of gas, and an outlet coupled directly or indirectly to the inlet to form a gas flow path, wherein the gas flow path comprises salt in solid form and wherein the inlet is arranged to direct an incoming a heated and humidified flow of gas to outlet to the patient's airway such that the flow of gas contacts the salt in the gas flow path.

Preferably the gas flow path comprises a conduit.

Preferably, in use, the inlet directs incoming heated and humidified flow of gas to contact a surface of the salt in solid form to uptake salt into the flow of gas.

Preferably the gas flow uptakes salt by elution.

Preferably upon uptake of salt, the heated and humidified flow of gas from the receptacle comprises eluted salt resulting in a salt laden gas.

Preferably the gas is heated to between 30-40 degrees C.

Preferably the flow rate of the gas is between 5-50 litres/minute.

Preferably the humidity of the gas is between 80%-100%.

Preferably the interface is one of a cannula and a tracheotomy connection.

Preferably the salt is in or proximate the outlet.

In another aspect the present invention may be said to consist in an interface for delivering a medicament to a patient, the interface comprising: a receptacle for the medicament in solid form, an inlet adapted to couple directly or indirectly to a source of a heated and humidified flow of gas, the inlet being arranged to direct an incoming heated and humidified flow of gas to the receptacle, and an outlet arranged to receive and deliver a heated and humidified flow of gas from the receptacle to the patient's airway.

In another aspect the present invention may be said to consist in an interface for delivering a medicament to a patient, the interface comprising: an inlet adapted to couple directly or indirectly to a source of a heated and humidified flow of gas, and an outlet coupled directly or indirectly to the inlet to form a gas flow path, wherein the gas flow path comprises medicament in solid form, and wherein the inlet is arranged to direct an incoming a heated and humidified flow of gas to outlet to the patient's airway such that the flow of gas contacts the medicament in the gas flow path.

In another aspect the present invention may be said to consist in a method of providing a medicament to a patient comprising: passing a humidified and heated flow of gas to contact the surface of a medicament in solid form disposed at or near a patient to uptake the medicament into the flow of gas, and delivering the medicament laden flow of gas to a patient.

In another aspect the present invention may be said to consist in a breathing assistance apparatus for delivering a medicament to a patient with a flow of gas comprising: a gases supply providing a flow of gas, a humidifier and heater for heating and humidifying a flow of gas from the gases supply, a patient interface according to claim 35 or 36 coupled to the gases supply via an inspiratory conduit to deliver a heated and humidified flow of gas from the gases supply to a patient.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

The term ‘comprising’ as used in this specification means ‘consisting at least in part of’, that is to say when interpreting statements in this specification which include that term, the features, prefaced by that term in each statement, all need to be present but other features can also be present.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### BRIEF DESCRIPTION OF THE DRAWINGS

Preferred forms of the present invention will now be described with reference to the accompanying drawings.

FIG. 1 is an illustration of a gases supply and humidifier system that may be used to provide a medicament to a patient.

FIG. 2 is a perspective view of an interface including a dosage receptacle in which to place a solid dose of a medicament.

FIG. 3 is a detailed view of the interface in FIG. 2.

FIG. 4 is a graph showing experimental data obtained by providing salt therapy according to the invention.

FIG. 5 is a detailed view of another nasal cannula interface.

FIG. 6 is a detailed view of a trachea interface (tracheotomy connection).

### DETAILED DESCRIPTION OF THE PREFERRED EMBODIMENT

### Overview

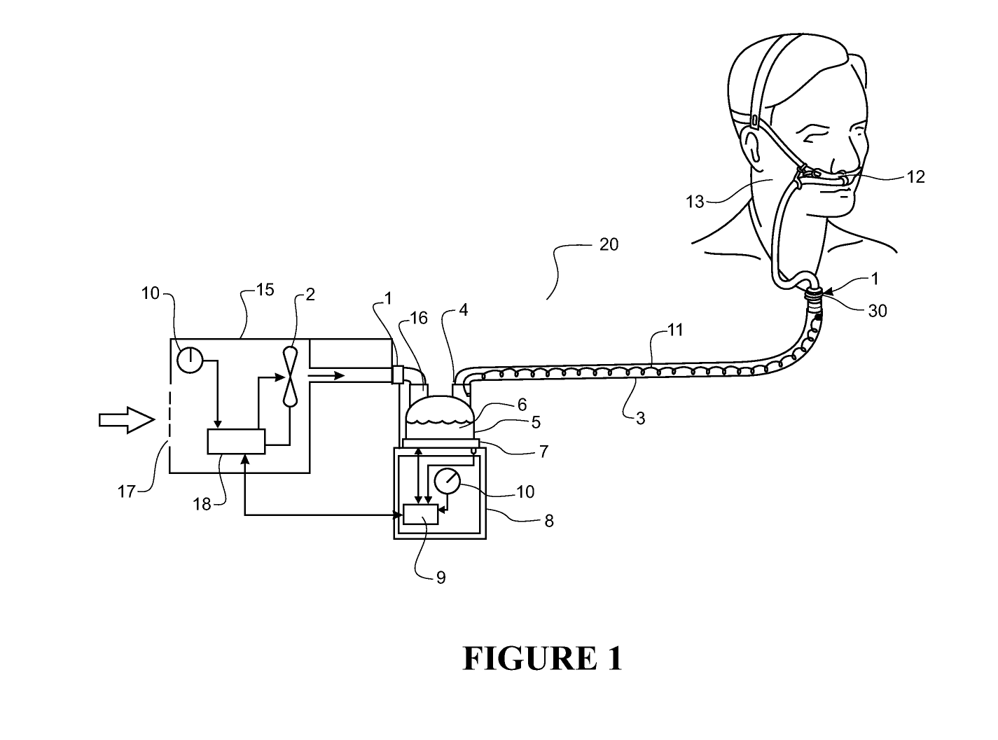

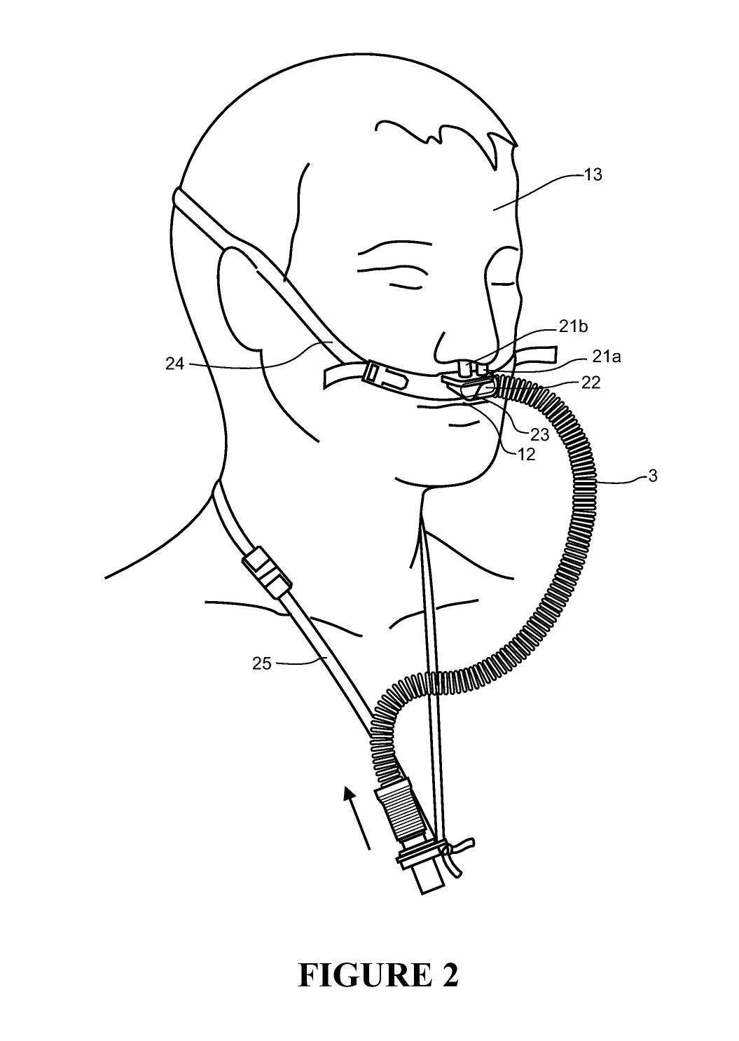

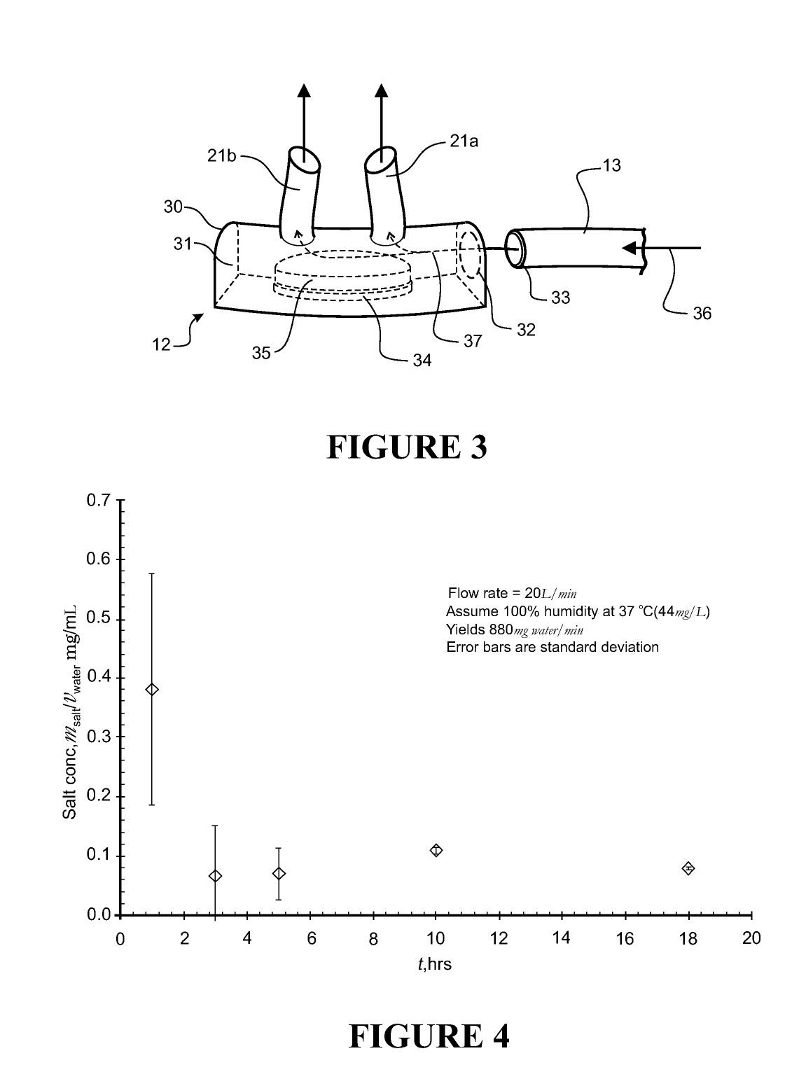

The present invention is an apparatus and method for delivering a gases flow with a medicament entrained in it. For example, the medicament could be salt such that the present invention delivers a salt-based therapy by way of non-invasive, positive pressure respiratory equipment. Direct interaction of a gases flow with a solid form of a medicament, either in a discrete form, such as in the general form of a tablet, or as a coating applied to a patient interface or an inspiratory conduit is described. The apparatus could be in the form of a gases supply and humidifying system with a patient interface and inspiratory conduit (as shown for example in FIG. 1). Alternatively, the apparatus could be an interface and/or conduit for use in conjunction with a gases supply and humidifying system (as shown for example in FIGS. 2 and 3).

The apparatus is adapted to provide medicament dosing by way of a receptacle or substrate for retaining medicament in solid form in a gases flow path to allow heated and humidified gas to contact the medicament (when retained in the receptacle or on the substrate) and uptake the medicament by elution into the gas flow to provide a medicament laden gas flow. For example, the medicament could be a solid tablet in the receptacle, or coated on the substrate. The gas contacts the medicament by passing across, over, or along a surface of or through the medicament. This medicament laden gas flow is directed to the patient. In the preferred embodiment where the medicament is salt, the apparatus facilitates a method of delivering salt-therapy to a patient.

The applicants have found that the inclusion of a soluble, porous, solid dosage form of medicament (for example, salts) placed in a gases flow results in significant delivery of a medicament in a form not observable by the naked eye (that is, not by macro-scale droplets or particles).

For example, exploratory trials conducted by the applicant have revealed that a solid dosage of sodium chloride (NaCl or commonly what is known as salt) in the form of a compressed powder in tablet form or similar, when placed within a nasal cannula, proximal to the outlet of the cannula, erodes (by elution) at a rate sufficient to deliver useful quantities of NaCl via the gases flow to the patient. No visible particles were observed to efflux from the cannula, but reduction in tablet mass and other qualitative tests suggest that the salt is transported by in the airflow and thus enters the patient respiratory tracts.

The invention can be used to provide, for example, salt therapy, cystic fibrosis therapy and/or chronic obstructive pulmonary disease therapy.

### Gases Supply and Humidifying System

FIG. 1 shows in general form a gases supply and humidifying system 20. The system forms the platform for apparatus for medicament dosing and can form part or all of an embodiment of the present invention, or alternatively be used in conjunction with an embodiment of the invention (such as an interface). A patient 13 receives humidified and pressurised gases through a patient interface 12 connected to a humidified gases transportation pathway or inspiratory conduit 3. The conduit 3 is connected to a humidifier 8 (including humidification chamber 5) supplied with gases from a blower 15 or other appropriate gases supply. The patient interface 13 used with the apparatus of the present invention may be a full-face mask, nasal mask, nasal cannula, oral mouthpiece or tracheotomy connection. In FIG. 1 a nasal cannula 12 is shown, by way of example.

The inspiratory conduit 3 is connected to the outlet 4 of the humidification chamber 5 that contains a volume of water 6. The humidification chamber 5 is preferably formed from a plastics material and may have a highly heat conductive base (for example an aluminium base) that is in direct contact with a heater plate 7 of humidifier 8. The humidifier 8 is provided with a controller 9 that may comprise a microprocessor based controller executing computer software commands stored in associated memory.

The controller 9 receives input from sources such as user input interface (such as a dial) 10 through which a user of the device may, for example, set a predetermined required value (preset value) of humidity or temperature of the gases supplied to patient 13. In response to the user set humidity or temperature value input via dial 10 and/or other possible inputs such as external or internal sensors that sense gases flow or temperature, or by parameters calculated in the controller, controller 9 determines when (or to what level) to energise heater plate 7 to heat the water 6 within the humidification chamber 5. As the volume of water 6 within the humidification chamber 5 is heated, water vapour begins to fill the volume of the chamber above the surface of the water. The water vapour is passed out of the humidification chamber outlet 4 with the flow of gases (for example air) provided from a gases supply means or blower 15. The gases from the blower 15 enter the humidification chamber 5 through the chamber inlet 16.

The blower 15 may be provided with a variable speed pump or fan 2 which draws air or other gases through the blower inlet 17. The speed of the variable speed pump or fan 2 may be controlled by a further controller 18 which responds either to inputs from controller 9 or to user-set predetermined required values (preset values) of pressure or fan speed, via dial or other user input interface 19. Additionally or alternatively, internal or external sensors may be used in the controlling of the pump or fan 2. Alternatively, the function of this controller 18 can be combined with the other controller 9.

The breathing assistance apparatus and humidification control may be that as described in PCT/NZ/000091 of Fisher & Paykel Healthcare Limited, the entire contents of which is incorporated by reference.

The gases supply and humidifying system can be an integrated system where the gases supply or blower is combined in one housing with the humidifier and humidifier chamber. For example, the humidifier unit may be mated to the blower unit by a slide-on or push connection, which ensures that the humidifier unit is rigidly connected to and held firmly in place on the main blower unit. An example of a system of this type is the Fisher and Paykel Healthcare Limited ‘slide-on’ water chamber system shown and described in U.S. Pat. No. 7,111,624, the entire contents of which is incorporated by reference. A variation of this design is a slide-on or clip-on design where the chamber is enclosed inside a portion of the integrated unit in use. An example of this type of design is described in WO 2004/112873 of Fisher & Paykel Healthcare Limited, the entire contents of which is incorporated by reference.

A heating element or wire 11 is preferably provided within, around and throughout the inspiratory conduit or tubing 3 to help prevent condensation of the humidified gases within the conduit. Such condensation is due to the temperature of the walls of the conduit being close to the ambient temperature, being the temperature of the surrounding atmosphere, which is usually lower than the temperature of the humidified gases within the conduit. The heater element effectively replaces the energy lost from the gases through conduction and convection during transit through the conduit. Thus the conduit heater element ensures the gases delivered are at an optimal temperature and humidity.

A heated tube with a heating wire such as that described in Fisher & Paykel Healthcare Limited's U.S. Pat. No. 6,078,730 (the entire contents of which is incorporated by reference) or any other similar tube and heating wire could be utilised with the present invention.

The gases supply and humidifying system 20 is used in conjunction with a medicament in solid form. In use, the heated and humidified gas flow is directed to contact with a medicament in solid form, so that the medicament is taken up in the gas flow and delivered to the patient. Preferably, the gas flow is heated to within a range of 30-40° C., and most preferably 35-37° C. The humidity of the gas flow is above 80% and preferably 100% or close to it. The flow rate is preferably between 5-50 litres per minute and preferably 10-30 litres per minute. It has been found that these ranges of heating, humidification and flow rate provide the most efficacious uptake of medicament, and delivery and absorption by the patient.

FIG. 4 shows experimental data for a test with a flow rate of 20 L/min, temperature of 37° C., humidity of 100%. The graph demonstrates the uptake of salt into the gas stream (measured as a concentration mg of salt per mL of water at different times.

As detailed above the gases flow to the patient is controlled such that the pressure, flow, temperature or humidity may be altered. Particularly, the gases flow may be altered to achieve a particular attrition/elution of the solid medicament. So that over a particular time period or at a particular particle rate the medicament is entrained into the gases flow and breathed in by the patient.

More particularly, the controlling of attrition of the solid medicament into the gases flow may include the controlling of: a) gases flow velocities, b) water vapour deposition and removal (using chemical and/or thermodynamic mechanisms), c) solid medicament dose substrate structure, or d) solid medicament dose morphology and microstructure (such as particle shape, porosity, inter-particle bonds, chemistry, formulation, and specific surface area).

### Cannula

In one embodiment, the interface 12 is a nasal cannula such as that shown in FIG. 2. It comprises an inlet (see FIG. 3) for coupling to the inspiratory conduit 3 and an outlet comprising two nasal prongs 21a, 21b for delivering gas received from the conduit 3 to the patient 13. The cannula 12 comprises a receptacle 22 for a medicament 23 in solid form. The cannula can be held in position via any suitable attachment, such as a head band 24. The conduit can be supported by a lanyard 25 around the patient's neck, for example.

FIG. 3 shows the nasal cannula 12 in more detail. The nasal cannula comprises a main housing 30 defining an internal volume 31. The main 30 housing comprises the inlet 32 on one side, adapted to couple to the inspiratory conduit 3 via a suitable friction or click fit coupling 33, or any other suitable coupling known in the art. The main housing 30 also comprises the, preferably two, nasal prongs 21a, 21b, which are tubular and open at both ends and extend from the main housing 30 creating a gas flow outlet from the interior of the main housing. The nasal prongs 21a, 21b form or comprise the outlet of the nasal cannula 12. The main housing 30 also defines or comprises a medicament receptacle 34, for retaining a medicament 35 in solid form.

The structure of the inlet 32, housing 30 and outlet 21a, 21b is such that gas flow 36 entering the inlet 32 via the conduit 3 is directed by the inlet and housing towards the receptacle 34, and then out the nasal prongs 21a, 21b to a patient's airway. The structure and arrangement of the inlet, housing and outlet form a flow path 37. In use, when the medicament 35 is placed in the receptacle 34 and the cannula 12 is connected to the gases supply/humidifier 20 via a conduit 3, the heated and humidified gas flow 36 entering the inlet 32 is directed to contact the medicament 35 in the receptacle 34, such that the gas flow 37 uptakes the medicament 35 by elution and the medicament laden gas flow 37 exits the outlet 21a, 21b to the patient's airway.

Alternatively, the medicament in solid form could be applied to a substrate, either in the conduit or in the nasal cannula. For example, the salt could coat the inside of the cannula or conduit.

In either embodiment, the medicament 35 in solid form should be positioned as near as possible to the patient, and preferably proximate, at or near the patient 13. This improves efficacy of delivery.

In other forms of the present invention, medicament dosage or entrainment of a medicament into a gases stream may be by the use of coated substrates which could form either an insert or part of the gases flow path components.

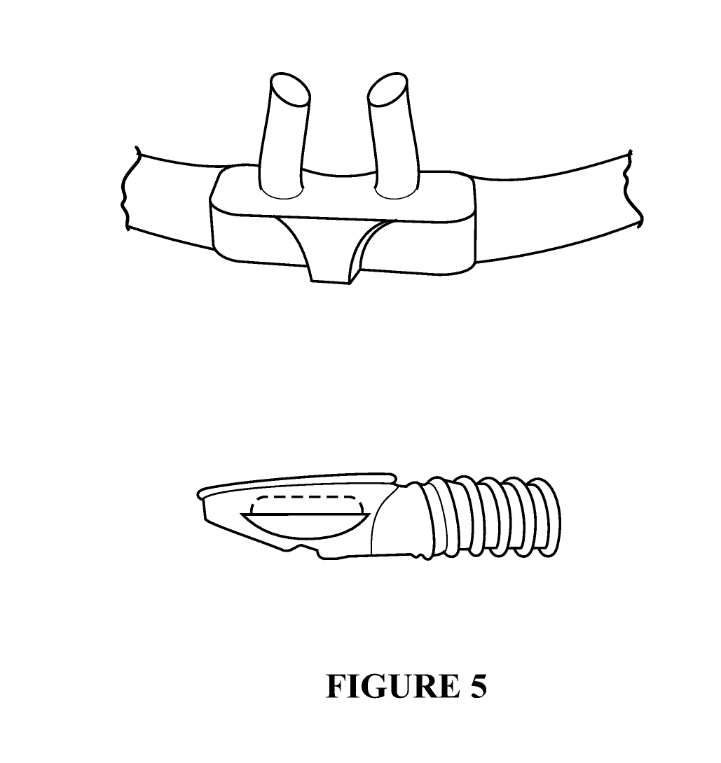

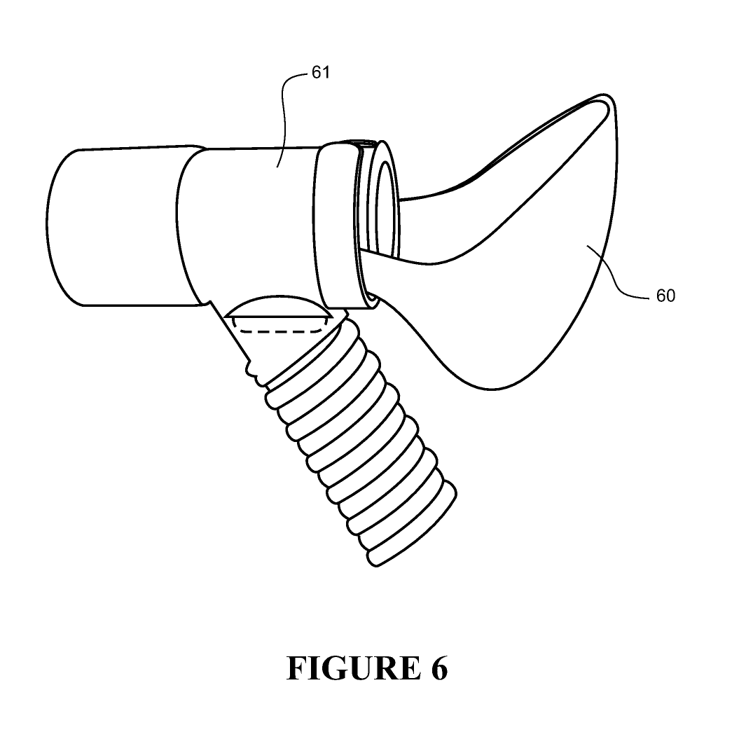

FIG. 5 shows another embodiment of a cannula that could be used.

FIG. 7 shows a tracheotomy connection 61, with an inlet shield 60.

### Advantages

The apparatus and method of the present invention provides a low cost method for delivering beneficial substances to the airways of a patient by way of elution of medicament using heated and humidified gas flow. Current treatments seem to be specialised in delivery format, requiring additional specialist equipment such as nebulisers. Also, current systems often require the use of sonication, metering or control systems.

The present apparatus and method does not require complex sonication, nebulisation metering, control or other such additional systems.

Further, the present invention provides a comfortable interface option for the patient. It is therefore more likely to be used systems for delivering treatments over sustained periods, thus providing advantages for low to high rate long term dosing available for use at home, even whilst the patient is at rest, sleeping, or in social situations.

Thus the present invention provides a passive delivery system of a medicament that requires no further apparatus, and as such can be fitted to existing respiratory treatment systems with ease.

1. An interface for delivering salt to a patient, the interface comprising
a receptacle for salt in solid form,
an inlet adapted to couple directly or indirectly to a source of a heated and humidified flow of gas, the inlet being arranged to direct an incoming heated and humidified flow of gas from the source of heated and humidified flow of gas to the receptacle, and
an outlet arranged to receive and deliver a heated and humidified flow of gas from the receptacle to the patient's airway.

2. An interface according to claim 1, wherein, in use, the receptacle contains salt in solid form and the inlet directs incoming heated and humidified flow of gas to contact a surface of the salt in solid form to uptake salt into the flow of gas.

3. An interface according to claim 2, wherein the flow of gas uptakes salt by elution.

4. An interface according to claim 2, wherein upon uptake of salt, the heated and humidified flow of gas from the receptacle comprises eluted salt resulting in a salt laden gas.

5. An interface according to claim 1, wherein the gas is heated to between 30-40 degrees C.

6. An interface according to claim 1, wherein the flow rate of the gas is between 5-50 litres/minute.

7. An interface according to claim 1, wherein the humidity of the gas is between 80%-100%.

8. An interface according to claim 1, wherein the interface is one of a cannula and a tracheostomy connection.

9. An interface according to claim 1, wherein the receptacle is in or proximate the outlet.

10. A method of providing salt therapy comprising:
passing a humidified and heated flow of gas to contact a surface of a salt in solid form disposed at or near a patient to uptake salt into the humidified and heated flow of gas, and delivering the salt laden flow of gas to a patient.

11. A method according to claim 1, wherein the method treats at least one of the group consisting of COPD and Cystic Fibrosis.

12. A method according to claim 10, wherein the gas is heated to between 30-40 degrees C.

13. A method according to claim 10, wherein the flow rate of the gas is between 5-50 litres/minute.

14. A method according to claim 10, wherein the humidity of the gas is between 80%-100%.

15. A method according to claim 10, wherein the gas flow uptakes salt by elution.

16. A breathing assistance apparatus for delivering salt therapy to a patient with a flow of gas comprising:
a gases supply providing a flow of gas,
a humidifier and heater adapted to heat and humidify a flow of gas from the gases supply,
a patient interface coupled to the gases supply via an inspiratory conduit to deliver a heated and humidified flow of gas from the gases supply to a patient, the patient interface comprising:
a receptacle for salt in solid form, an inlet adapted to couple directly or indirectly to a source of a heated and humidified flow of gas,
the inlet being arranged to direct an incoming heated and humidified flow of gas from the source of heated and humidified flow of gas to the receptacle, and
an outlet arranged to receive and deliver a heated and humidified flow of gas from the receptacle to the patient's airway.

17. A breathing assistance apparatus according to claim 16, wherein, in use, the receptacle contains salt in solid form and the inlet directs incoming heated and humidified flow of gas to contact a surface of the salt in solid form to uptake salt into the flow of gas.

18. A breathing apparatus according to claim 16, wherein the gas flow uptakes salt by elution.

19. A breathing assistance apparatus according to claim 16, wherein upon uptake of salt, the heated and humidified flow of gas from the receptacle comprises eluted salt resulting in a salt laden gas.

20. A breathing apparatus according to claim 16, wherein the gas is heated to between 30-40 degrees C.

21. A breathing apparatus according to claim 16, wherein the flow rate of the gas is between 5-50 litres/minute.

22. A breathing assistance apparatus according to claim 16, wherein the humidity of the gas is between 80%-100%.

23. A breathing assistance apparatus according to claim 16, wherein the interface is one of a cannula or a tracheotomy connection.

24. A breathing assistance apparatus according to claim 16, wherein the receptacle is in or proximate the outlet.

25. An interface for delivering salt to a patient, the interface comprising
an inlet adapted to couple directly or indirectly to a source of a heated and humidified flow of gas, and
an outlet coupled directly or indirectly to the inlet to form a gas flow path,
wherein the gas flow path comprises salt in solid form and wherein the inlet is arranged to direct an incoming a heated and humidified flow of gas to outlet to the patient's airway such that the flow of gas contacts the salt in the gas flow path.

26. An interface according to claim 25, wherein the gas flow path comprises a conduit.

27. An interface according to claim 25, wherein, in use, the inlet directs incoming heated and humidified flow of gas to contact a surface of the salt in solid form to uptake salt into the flow of gas.

28. An interface according to claim 25, wherein the gas flow uptakes salt by elution.

29. An interface according to claim 25, wherein upon uptake of salt, the heated and humidified flow of gas from the receptacle comprises eluted salt resulting in a salt laden gas.

30. An interface according to claim 25, wherein the gas is heated to between 30-40 degrees C.

31. An interface according to claim 25, wherein the flow rate of the gas is between 5-50 litres/minute.

32. An interface according to claim 25, wherein the humidity of the gas is between 80%-100%.

33. An interface according to claim 25, being one of a cannula and a tracheotomy connection.

34. An interface according to claim 25, wherein the salt is in or proximate the outlet.

35. An interface for delivering a medicament to a patient, the interface comprising
a receptacle for the medicament in solid form,
an inlet adapted to couple directly or indirectly to a source of a heated and humidified flow of gas, the inlet being arranged to direct an incoming heated and humidified flow of gas to the receptacle, and
an outlet arranged to receive and deliver a heated and humidified flow of gas from the receptacle to the patient's airway.

36. An interface for delivering a medicament to a patient, the interface comprising
an inlet adapted to couple directly or indirectly to a source of a heated and humidified flow of gas, and
an outlet coupled directly or indirectly to the inlet to form a gas flow path,
wherein the gas flow path comprises medicament in solid form and wherein the inlet is arranged to direct an incoming a heated and humidified flow of gas to outlet to the patient's airway such that the flow of gas contacts the medicament in the gas flow path.

37. A method of providing a medicament to a patient comprising:
passing a humidified and heated flow of gas to contact the surface of a medicament in solid form disposed at or near a patient to uptake the medicament into the flow of gas, and delivering the medicament laden flow of gas to a patient.

38. A breathing assistance apparatus for delivering a medicament to a patient with a flow of gas, comprising:
a gases supply providing a flow of gas,
a humidifier and heater for heating and humidifying a flow of gas from the gases supply,
a patient interface according to claim 35 coupled to the gases supply via an inspiratory conduit to deliver a heated and humidified flow of gas from the gases supply to a patient.

39. A breathing assistance apparatus for delivering a medicament to a patient with a flow of gas comprising:
a gases supply providing a flow of gas,
a humidifier and heater for heating and humidifying a flow of gas from the gases supply,
a patient interface according to claim 36 coupled to the gases supply via an inspiratory conduit to deliver a heated and humidified flow of gas from the gases supply to a patient.
